(19) United States
(12) Patent Application Publication
(10) Pub. No.: US 2023/0321326 A1
(43) Pub. Date: Oct. 12, 2023

(54) CATHETER WITH INHERENT ANTIMICROBIAL PROPERTIES

(71) Applicant: Becton, Dickinson and Company, Franklin Lakes, NJ (US)

(72) Inventors: Amit Limaye, Wayne, NJ (US); Margaret Taylor, Groton, MA (US)

(21) Appl. No.: 17/718,069

(22) Filed: Apr. 11, 2022

(51) Int. Cl. A61L 29/06; A61L 29/16; A61L 29/08; B29D 23/00; C08L 67/04

(52) U.S. Cl. CPC A61L 29/06; A61L 29/16; A61L 29/085; B29D 23/001; C08L 67/04; C08L 2203/02

(57) ABSTRACT

A catheter configured to provide inherent antimicrobial properties includes a polyhydroxyalkanoate (PHA). In some embodiments, an extruded catheter body is fabricated of a PHA. In some embodiments, an extruded catheter body has an exterior layer containing PHA, an interior layer containing PHA, or both exterior and interior layers containing PHA. The PHA layer may be co-extruded with the catheter body. The PHA layer may be coating applied to the catheter body. Useful PHA materials to provide inherent antimicrobial properties include poly-4-hydroxybutyrate (P4HB) and copolymers of P4HB. The catheter may be an IV catheter, a urinary catheter, a dialysis catheter, or any other catheter introduced into a body lumen.

### BACKGROUND

The current invention relates to catheters comprising a polyhydroxyalkanoate (PHA) to provide inherent antimicrobial properties. The current invention also relates to catheters coated with a polyhydroxyalkanoate to provide inherent antimicrobial properties.

IV catheters are life saving devices that have become a standard of care. For example, peripheral intravenous catheters (PIVCs) are often used in acute applications such as short-inpatient and outpatient services. Alternatively, peripherally inserted central catheters (PICCs) are used in chronic/long-duration applications.

Unfortunately, IV access lines are also associated with a high incidence of central line-associated bloodstream infection (CLABSI) and catheter-related bloodstream infection (CRBSI), which are infections resulting due to placement of these catheters into the blood stream. These infections are an important cause of illness and excess medical costs, as approximately 250.000-400,000 cases of central venous catheter (CVC) associated bloodstream infections occur annually in US hospitals. In addition to the monetary costs, these infections are associated with anywhere from 20,000 to 100,000 deaths each year. Despite guidelines to help reduce healthcare associated infections (HAIs), catheter-related bloodstream infections continue to plague our healthcare system.

Multiple approaches are utilized to mitigate the occurrence of these infections—namely proper insertion site cleaning, good catheter placement practice, and use of antimicrobial agents in or on the catheter tubing to suppress microbial growth.

A majority of the commercially available IV catheters that are used today do not have any anti-microbial action.

To provide antimicrobial properties, anti-microbial agents need to be immobilized into the matrix or coated onto the catheter surface. These catheters, however, have given less than satisfactory results.

The microbial agents typically targeted for use in these applications, such silver and chlorhexidine gluconate (CHG), have toxicological implications and hence the dose and rate of elution needs to be managed carefully.

There are multiple known instances and some patient profiles showing sensitivity to CHG. This includes pediatric and neonatal applications.

Microbial resistance to CHG, while limited, has been reported. This limits the efficacy of using the chlorhexidine class of antimicrobials for future applications.

One solution to the above limitations is use of other microbicidal agents such Anti-Microbial Peptide (AMP) or Host Defense Peptide (HDP) analogs. These analogs belong in the same class as peptide molecules produced by the body and hence do not elicit an immune or toxicological response. However, these AMP analogs are either difficult to make or can be made only in small quantities. Alternatively other mimetics of host body response may also be used to impart antimicrobial activity. In both cases, these molecules need to be coated onto or incorporated into the bulk of the catheter tubing, resulting in process steps that need to take into account the special requirements of the substrate material and the fragile nature of these analogs—and this in turn impacts the resulting elution profile.

More than 3000 AMPs have been discovered, 7 of which have been approved by FDA and commercialized (Reference: Antibiotics 2020, 9, 24). Multiple companies and universities are investigating novel applications of AMPS. Technologies using AMP analogs include molecules that are either inspired by naturally occurring peptide molecules or are synthetic non-peptide small molecule mimetics of endogenous host defense or antimicrobial peptides. Examples of technology companies include Peptilogics, Allvivo, Riptide Bioscience, Amprologix, Demegen, AMPbiotech and ContraFect.

Accordingly, there is a need in the art for catheters having improved antimicrobial capabilities. Such method and systems are disclosed herein.

The subject matter claimed herein is not limited to embodiments that solve any disadvantages or that operate only in environments such as those described above. Rather, this background is only provided to illustrate one example technology area where some implementations described herein may be practiced.

### SUMMARY

The present invention has been developed in response to problems and needs in the art that have not yet been fully resolved by currently available antimicrobial catheters. The disclosed catheters provide inherent antimicrobial properties.

One general aspect includes a catheter with inherent antimicrobial properties having an extruded catheter body which includes a thermoplastic polyhydroxyalkanoate polymer.

Implementations may include one or more of the following features. The thermoplastic polyhydroxyalkanoate polymer may be poly-4-hydroxybutyrate. The thermoplastic polyhydroxyalkanoate polymer may be a poly-4-hydroxybutyrate copolymer. The catheter body may be fabricated of poly-4-hydroxybutyrate. The catheter body may be fabricated of a poly-4-hydroxybutyrate copolymer. The catheter body may include a co-extruded layer of thermoplastic polyhydroxyalkanoate polymer. The thermoplastic polyhydroxyalkanoate polymer may be a poly-4-hydroxybutyrate copolymer. The catheter may be a peripheral intravenous catheter (PIVC). The catheter may be a peripherally inserted central catheter (PICC). The catheter may be a urinary catheter. The catheter may be a dialysis catheter, including acute and chronic dialysis catheters, and peritoneal dialysis catheters. The catheter may be any other catheter introduced into a body lumen.

Another general aspect includes a method of manufacturing a catheter with inherent antimicrobial properties. The method includes obtaining a thermoplastic polyhydroxy-alkanoate polymer. The method also includes extruding the thermoplastic polyhydroxyalkanoate polymer to form an elongate catheter body having one or more lumens extending through a portion of the elongate catheter body.

Implementations may include one or more of the following features. In the method, the thermoplastic polyhydroxyalkanoate polymer may be poly-4-hydroxybutyrate. The thermoplastic polyhydroxyalkanoate polymer may be a poly-4-hydroxybutyrate copolymer.

Another general aspect includes a method of manufacturing a catheter with inherent antimicrobial properties. The method includes extruding an elongate catheter body having one or more lumens extending through a portion of the elongate catheter body. The method also includes co-extruding a thermoplastic polyhydroxyalkanoate polymer layer bonded to the catheter body. Alternatively, the method also includes coating the catheter body with a polyhydroxyalkanoate polymer coating.

Implementations may include one or more of the following features. The thermoplastic polyhydroxyalkanoate polymer may be poly-4-hydroxybutyrate. The thermoplastic polyhydroxyalkanoate polymer may be a poly-4-hydroxybutyrate copolymer. The thermoplastic polyhydroxyalkanoate polymer layer may include poly-4-hydroxybutyrate. The thermoplastic polyhydroxyalkanoate polymer layer may include a poly-4-hydroxybutyrate copolymer.

It is to be understood that both the foregoing general description and the following detailed description are exemplary and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is a perspective view of an extruded catheter body.

FIG. 2A is a cross-sectional representation of a portion of a catheter body having an outer layer or coating which contains a polyhydroxyalkanoate (PHA) to provide antimicrobial properties.

FIG. 2B is a cross-sectional representation of a portion of a catheter body having an inner layer or coating which contains a polyhydroxyalkanoate (PHA) to provide antimicrobial properties.

FIG. 2C is a cross-sectional representation of a portion of a catheter body having outer and inner layers or coatings which contain a polyhydroxyalkanoate (PHA) to provide antimicrobial properties.

### DESCRIPTION OF EMBODIMENTS

The disclosure relates to a catheter containing a polyhydroxyalkanoate (PHA). The disclosure relates to a catheter having a layer containing a polyhydroxyalkanoate (PHA). The PHA provides antimicrobial properties. The disclosure further relates to methods of manufacturing a catheter containing a PHA configured to provide antimicrobial properties. The disclosure further relates to methods of manufacturing a catheter having a coating containing a PHA configured to provide antimicrobial properties.

Polyhydroxyalkanoates (PHA) are thermoplastic polyesters produced in nature by numerous microorganisms. See, for example, Steinbüchel A., et al. Diversity of Bacterial Polyhydroxy-alkanoic Acids, FEMS Microbial. Lett., 128: 219-228 (1995)). They can be processed by traditional polymer processing techniques. Over one hundred different monomers can be combined within this family to give materials with different properties. Polyhydroxyalkanoates include homopolymers, copolymers, terpolymers, and other polymers containing mixtures of different PHA monomers. Non-limiting examples of polyhydroxyalkanoates include poly-3-hydroxybutyrate (PHB), poly-3-hydroxyvalerate (PHV), poly-(R)-3-hydroxybutyrate-co-(R)-3-hydroxyvalerate (PHBV), poly-4-hydroxybutyrate (P4HB), and poly-4-hydroxybutyrate copolymers.

Poly-4-hydroxybutyrate copolymers, as used herein, means any polymer of 4-hydroxybutyrate with one or more different hydroxy acid units. One non-limiting poly-4-hydroxybutyrate copolymer is a copolymer of poly-4-hydroxybutyrate and poly-3-hydroxybutyrate.

PHB and P4HB possess different physical properties. A range of PHA copolymers containing 4-hydroxybutyrate and poly-3-hydroxybutyrate can be prepared with a range of intermediate properties between those of PHB and P4HB.

P4HB has a long clinical history of use in blood contact applications. It has been approved by the U.S. Food and Drug Administration for use in hernia mesh applications under the brand name Phasix® owned by Becton, Dickinson and Company.

It has been found that P4HB provides antimicrobial activity. P4HB exhibits the unique ability to promote endogenous antimicrobial peptide expression by macrophages in the immune system of the host. See, 4-Hydroxybutyrate Promotes Endogenous Antimicrobial Peptide Expression in Macrophages, Tissue Engineering Part A Vol. 25, No. 9-10, 2018; Role of 4-hydroxybutyrate in increased resistance to surgical site infections associated with surgical meshes, Biomaterials, Vol. 267, January 2021, 120493.

Unlike other bio-derived polymers, PHA polymers, including P4HB can be easily extruded and formed into various configurations such as tubing, mesh, foam, fibers and plugs.

Similar to fossil-fuel derived polymer materials, the mechanical properties of PHA polymers can be modulated by changing the molecular weight. This imparts the ability to make catheter tubing of different physical properties such as tensile strength and hardness requirements. As noted, PHA polymers may be made as co-polymers.

P4HB is bio-absorbable over long periods of time (1.5-2 years), but still maintains excellent mechanical properties as a function of time.

In some embodiments, the catheter is made by extruding a PHA polymer co-polymerized with one or more catheter substrate polymers to provide a catheter body having antimicrobial activity.

The catheter may be configured with a PHA polymer layer on an interior surface, an exterior surface, or interior and exterior surfaces of the catheter body.

In some embodiments the PHA polymer layer is prepared by co-extruding a PHA polymer material with another polymeric material configured to form the catheter body.

Co-extrusion is a useful technique to prepare multilayer catheter tubing. For instance, the catheter body may be extruded using a catheter body polymeric material having desired physical or mechanical characteristics. The layer of PHA polymer may be co-extruded with the catheter body polymeric material so that a catheter body having a PHA polymer layer is manufactured in one process step.

Non-limiting examples of typical catheter body polymeric materials include common thermoplastic elastomers, such as polyethylene (PE), including low density polyethylene (“LDPE”), linear low density polyethylene (“LLDPE”), high density polyethylene (“HDPE”) and blends thereof, polypropylene (PP), polyvinyl chloride (PVC), polyurethane (TPU), polytetrafluoroethylene (PTFE), and ethylene vinyl acetate (EVA).

A tie layer or bonding layer may be provided to anchor, join, or immobilize the two co-extruded layers and prevent delamination. A tie layer or bonding layer may mimic chemical properties of the polymeric materials used to co-extrude the layers to facilitate bonding of the polymeric materials. This is particularly helpful when the polymeric materials used to co-extrude the layers are chemically dissimilar.

In some embodiments, the PHA polymer layer is prepared by coating the catheter body.

In some embodiments the coating is selected from a dip coating and a spray coating.

In some embodiments the coating step is accomplished by dip coating the catheter extrusion in a polymer solution comprising a polyhydroxyalkanoate (PHA) composition.

Known coating technologies which incorporate the antimicrobial compound may be used. Examples of such coating technologies includes, but are not limited to, a dip coating, a spray coating, an imbibe coating, and a hydrogel coating. In some embodiments a primer chemistry may be used to improve coating adhesion.

Non-limiting examples of solvents which may be used with PHA-based coatings include methanol, ethanol, isopropyl alcohol (IPA), dioxolane, methyl ethyl ketone (MEK), tetrahydrofuran (THF), and acetone. The coating solutions typically dissolve at temperatures in the range of 50 to 80° C. The dip coating and spray coating process is typically performed at room temperature.

The PHA materials typically have a molecular weight over 300, for example between 300 and 10⁷. In one exemplary embodiment the PHA polymers have a molecular weight in the range of 1000 to 1,500,000 Daltons. In an embodiment, the PHA polymers have a molecular weight in the range of 10,000 to 1,000,000 Daltons. In an embodiment, the polymers have a molecular weight in the range of 50,000 to 500,000 Daltons. In an embodiment, the PHA polymers have a molecular weight in the range of 100,000 to 300,000 Daltons. In an embodiment, the PHA polymers have a molecular weight of about, 100, 1000, 10,000, 20,000, 30,000 40,000, 50,000, 60,000, 70,000, 80,000, 90,000, 100,000, 150,000, 200,000, 250,000, 300,000, 350,000, 400,000, 450,000, 500,000, 555,000, 600,000, 650,000, 700,000, 750,000, 800,000, 850,000, 900,000, 950,000, 1,000,000, 1,500,000, 2,000,000, 2,500,000, 3,000,000, 4,000,000, 5,000,000, 6,000,000, 7,000,000, 8,000,000, 9,000,000, or 10,000,000 Daltons, where any of the stated values can form an upper or lower endpoint of a range.

The PHA materials may contain or be modified to include other materials used to modify the mechanical properties of PHAs such as plasticizers, filters, nucleating agents, colorants, stabilizers, modifiers and binders.

The resulting catheter manufactured as described above is configured to provide antimicrobial properties.

The disclosed catheters may be used in a variety of different medical applications. In some embodiments, the catheter is a peripheral intravenous catheter (PIVC). In some embodiments, the catheter is a peripherally inserted central catheter (PICC).

In some embodiments, the catheter is a urinary catheter. Non-limiting examples of urinary catheters include indwelling urinary catheters such as Foley catheters; external catheters such as the PureWick™ female external catheter from C. R. Bard, Inc.; and intermittent urinary catheters, with and without lubricious coatings, including the Magic³ GO™ intermittent catheter also from C. R. Bard, Inc.

In some embodiments, the catheter is a dialysis catheter, including acute and chronic dialysis catheters, and peritoneal dialysis catheters. In some embodiments, the catheter is a catheter introduced into a body lumen.

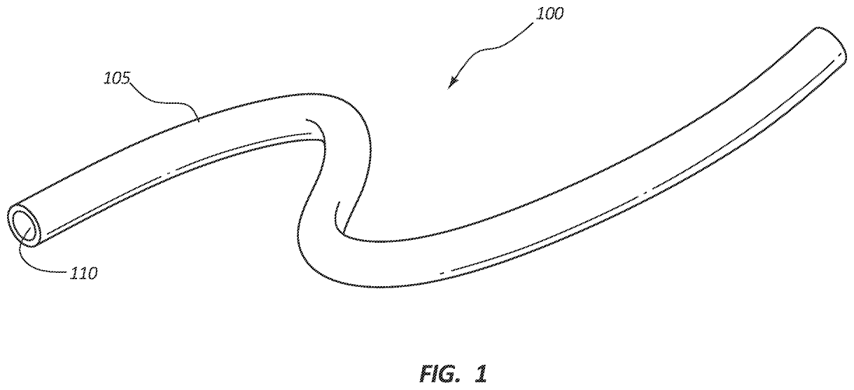

FIG. 1 depicts a portion of a catheter 100 fabricated of a polyhydroxyalkanoate composition selected to provide antimicrobial properties. As shown therein, at least a portion of the catheter 100 may be formed by extrusion of a polymeric matrix including polyhydroxyalkanoate composition. The catheter 100 includes an extruded catheter body 105 comprising a polyhydroxyalkanoate polymer. As shown in FIG. 1 the catheter 100 may also include a lumen 110 extending through at least a portion of the catheter body 105. While not shown in FIG. 1, the catheter 100 may include more than one lumen extending through at least a portion of the catheter body 105.

It will further be appreciated that the shape and/or size of the catheter 100 can be varied as desired. For example, various shapes and/or sizes of extrusion dies can be used to form catheters having particular shapes and/or sizes. Accordingly, it will be understood that the embodiment of FIG. 1 is merely exemplary of one type of extruded catheter.

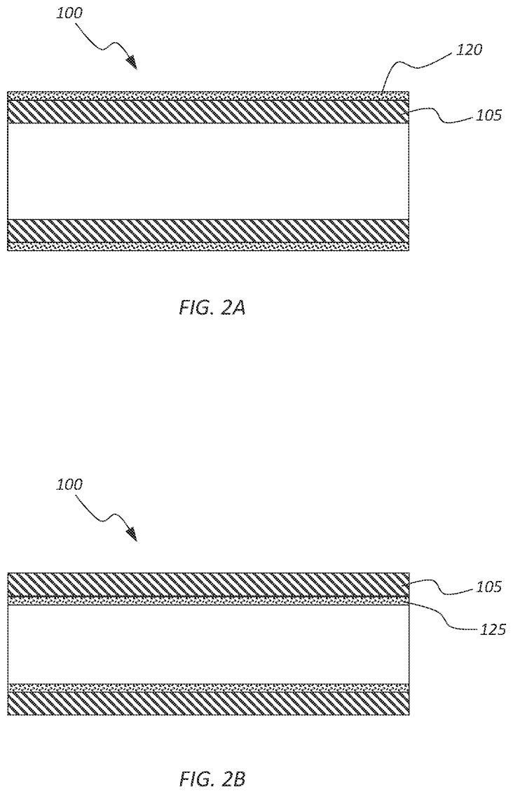

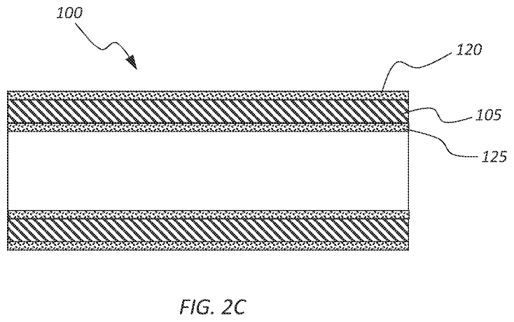

FIG. 2A is a cross-sectional representation of portion of a catheter 100 having a catheter body 105 fabricated of conventional polymer material. The catheter further includes a layer 120 on the catheter body 105 comprising a polyhydroxyalkanoate polymer. The layer 120 may comprise P4HB. The layer 120 may comprise a copolymer comprising P4HB. In the embodiment shown in FIG. 2A the layer 120 is disposed on an outer surface of the catheter body 105.

The layer 120 may be made by a co-extrusion process. The layer 120 may be made by a coating process.

FIG. 2B is a cross-sectional representation of portion of a catheter 100 having a catheter body 105 fabricated of conventional polymer material. The catheter further includes a layer 125 on the catheter body 105 comprising a polyhydroxyalkanoate polymer. The layer 125 may comprise P4HB. The layer 125 may comprise a copolymer comprising P4HB. In the embodiment shown in FIG. 2B the layer 125 is disposed on an inner surface of the catheter body 105.

The layer 125 may be made by a co-extrusion process. The layer 125 may be made by a coating process.

FIG. 2C is a cross-sectional representation of portion of a catheter 100 having a catheter body 105 fabricated of conventional polymer material. The catheter further includes a layer 120 on the catheter body 105 comprising a polyhydroxyalkanoate polymer. The layer 120 may comprise P4HB. The layer 120 may comprise a copolymer comprising P4HB. The catheter further includes a layer 125 on the catheter body 105 comprising a polyhydroxyalkanoate polymer. The layer 125 may comprise P4HB. The layer 125 may comprise a copolymer comprising P4HB. In the embodiment shown in FIG. 2C the layer 120 is disposed on an outer surface of the catheter body 105. In the embodiment shown in FIG. 2C the layer 125 is disposed on an inner surface of the catheter body 105.

The layers 120, 125 may be made by a co-extrusion process. The layers 120, 125 may be made by a coating process.

### EMBODIMENTS

Various embodiments are listed below. It will be understood that the embodiments listed below may be combined with all aspects and other embodiments in accordance with the scope of the invention.

Embodiment 1. A catheter with inherent antimicrobial properties comprising an extruded catheter body comprising a thermoplastic polyhydroxyalkanoate polymer.

Embodiment 2. The catheter of embodiment 1, wherein the thermoplastic polyhydroxyalkanoate polymer is poly-4-hydroxybutyrate.

Embodiment 3. The catheter of embodiment 1, wherein the thermoplastic polyhydroxyalkanoate polymer is a poly-4-hydroxybutyrate copolymer.

Embodiment 4. The catheter of embodiment 1, wherein the catheter body consists of poly-4-hydroxybutyrate.

Embodiment 5. The catheter of embodiment 1, wherein the catheter body consists of a poly-4-hydroxybutyrate copolymer.

Embodiment 6. The catheter of embodiment 1, wherein the catheter body comprises a co-extruded layer of thermoplastic polyhydroxyalkanoate polymer.

Embodiment 7. The catheter of embodiment 6, wherein the thermoplastic polyhydroxyalkanoate polymer is a poly-4-hydroxybutyrate copolymer.

Embodiment 8. The catheter of embodiment 1, wherein the catheter body comprises a coating of thermoplastic polyhydroxyalkanoate polymer.

Embodiment 9. The catheter of any preceding embodiment, wherein the catheter is a peripheral intravenous catheter (PIVC).

Embodiment 10. The catheter of any preceding embodiment, wherein the catheter is a peripherally inserted central catheter (PICC).

Embodiment 11. The catheter of any preceding embodiment, wherein the catheter is a urinary catheter.

Embodiment 12. The catheter of any preceding embodiment, wherein the catheter is a dialysis catheter.

Embodiment 13. A method of manufacturing a catheter with inherent antimicrobial properties, comprising: obtaining a thermoplastic polyhydroxyalkanoate polymer; and extruding the thermoplastic polyhydroxyalkanoate polymer to form an elongate catheter body having one or more lumens extending through a portion of the elongate catheter body.

Embodiment 14. The method of embodiment 13, wherein the thermoplastic polyhydroxyalkanoate polymer is poly-4-hydroxybutyrate.

Embodiment 15. The method of embodiment 13, wherein the thermoplastic polyhydroxyalkanoate polymer is a poly-4-hydroxybutyrate copolymer.

Embodiment 16. The method of embodiment 13, wherein the thermoplastic polyhydroxyalkanoate polymer consists of poly-4-hydroxybutyrate.

Embodiment 17. The method of embodiment 13, wherein the thermoplastic polyhydroxyalkanoate polymer consists of poly-4-hydroxybutyrate copolymer.

Embodiment 18. A method of manufacturing a catheter with inherent antimicrobial properties, comprising: extruding an elongate catheter body having one or more lumens extending through a portion of the elongate catheter body; and co-extruding a thermoplastic polyhydroxyalkanoate polymer layer bonded to the catheter body.

Embodiment 19. The method of embodiment 18, wherein the thermoplastic polyhydroxyalkanoate polymer is poly-4-hydroxybutyrate.

Embodiment 20. The method of embodiment 18, wherein the thermoplastic polyhydroxyalkanoate polymer is a poly-4-hydroxybutyrate copolymer.

Embodiment 21. The method of embodiment 20, wherein the thermoplastic polyhydroxyalkanoate polymer layer consists of poly-4-hydroxybutyrate.

Embodiment 22. The method of embodiment 18, wherein the thermoplastic polyhydroxyalkanoate polymer layer consists of a poly-4-hydroxybutyrate copolymer.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention. It should be understood that the embodiments may be combined.

1. A catheter with inherent antimicrobial properties comprising an extruded catheter body comprising a thermoplastic polyhydroxyalkanoate polymer.

2. The catheter of claim 1, wherein the thermoplastic polyhydroxyalkanoate polymer is poly-4-hydroxybutyrate.

3. The catheter of claim 1, wherein the thermoplastic polyhydroxyalkanoate polymer is a poly-4-hydroxybutyrate copolymer.

4. The catheter of claim 1, wherein the catheter body consists of poly-4-hydroxybutyrate.

5. The catheter of claim 1, wherein the catheter body consists of a poly-4-hydroxybutyrate copolymer.

6. The catheter of claim 1, wherein the catheter body comprises a co-extruded layer of thermoplastic polyhydroxyalkanoate polymer.

7. The catheter of claim 6, wherein the thermoplastic polyhydroxyalkanoate polymer is a poly-4-hydroxybutyrate copolymer.

8. The catheter of claim 1, wherein the catheter body comprises a coating of thermoplastic polyhydroxyalkanoate polymer.

9. The catheter of claim 1, wherein the catheter is a peripheral intravenous catheter (PIVC).

10. The catheter of claim 1, wherein the catheter is a peripherally inserted central catheter (PICC).

11. The catheter of claim 1, wherein the catheter is a urinary catheter.

12. The catheter of claim 1, wherein the catheter is a dialysis catheter.

13. A method of manufacturing a catheter with inherent antimicrobial properties, comprising:
obtaining a thermoplastic polyhydroxyalkanoate polymer; and
extruding the thermoplastic polyhydroxyalkanoate polymer to form an elongate catheter body having one or more lumens extending through a portion of the elongate catheter body.

14. The method of claim 13, wherein the thermoplastic polyhydroxyalkanoate polymer is poly-4-hydroxybutyrate.

15. The method of claim 13, wherein the thermoplastic polyhydroxyalkanoate polymer is a poly-4-hydroxybutyrate copolymer.

16. The method of claim 13, wherein the thermoplastic polyhydroxyalkanoate polymer consists of poly-4-hydroxybutyrate.

17. The method of claim 13, wherein the thermoplastic polyhydroxyalkanoate polymer consists of poly-4-hydroxybutyrate copolymer.

18. A method of manufacturing a catheter with inherent antimicrobial properties, comprising:
extruding an elongate catheter body having one or more lumens extending through a portion of the elongate catheter body; and
co-extruding a thermoplastic polyhydroxyalkanoate polymer layer bonded to the catheter body.

19. The method of claim 18, wherein the thermoplastic polyhydroxyalkanoate polymer is poly-4-hydroxybutyrate.

20. The method of claim 18, wherein the thermoplastic polyhydroxyalkanoate polymer is a poly-4-hydroxybutyrate copolymer.

21. The method of claim 18, wherein the thermoplastic polyhydroxyalkanoate polymer layer consists of poly-4-hydroxybutyrate.

22. The method of claim 18, wherein the thermoplastic polyhydroxyalkanoate polymer layer consists of a poly-4-hydroxybutyrate copolymer.
